(19) United States
(12) Patent Application Publication
(10) Pub. No.: US 2018/0360005 A1
(43) Pub. Date: Dec. 20, 2018

(54) TRPV1 KNOCK-IN METHOD AND TRPV1 MICE FOR ADIPOSE TISSUE MODULATION

(71) Applicant: UNIVERSITY OF WYOMING, Laramie, WY (US)

(72) Inventors: Baskaran THYAGARAJAN, Laramie, WY (US); Padmamalini BASKARAN, Laramie, WY (US)

(21) Appl. No.: 16/010,303

(22) Filed: Jun. 15, 2018

Related U.S. Application Data
(60) Provisional application No. 62/521,075, filed on Jun. 16, 2017.

(51) Int. Cl. A01K 67/027; C12N 15/85

(52) U.S. Cl. CPC A01K 67/0276; C12N 15/8509; A01K 2267/0306; C12N 2015/859; A01K 2267/0393; C12N 2015/8536; A01K 2227/105

(57) ABSTRACT

In an embodiment, a transgenic mouse is provided. The transgenic mouse's genetic material includes a Cre mouse TRPV1 protein knockout and a rat TRPV1 cyanreporter protein expressed in a tissue. In another embodiment, a method for producing a conditional TRPV1 knock-in is provided. The method includes mating a Cre mouse with a mouse expressing a rat TRPV1 and cyanreporter protein resulting in a first progeny comprising mice heterozygous for a Cre allele and heterozygous for a rat TRPV1 and cyanreporter allele; mating the first heterozygous mouse with a mouse TRPV1 knockout resulting in a second progeny; mating the second progeny with a mouse TRPV1 knockout; and obtaining a third progeny comprising mice heterozygous for the TRPV1 knockout allele, the Cre allele, and the rat TRPV1 and cyanreporter allele.

### CROSS REFERENCE TO RELATED APPLICATIONS

This application claims benefit of U.S. Provisional Patent Application No. 62/521,075, filed Jun. 16, 2017, the entirety of which is herein incorporated by reference.

### FIELD

Embodiments of the present disclosure generally relate to genetically modified mammals.

### BACKGROUND

Obesity is associated with serious health risks including metabolic dysfunction, type 2 diabetes, dyslipidemia, hypertension, and cardiovascular diseases. Worldwide, two billion adults and children are overweight and one third are obese, with these numbers rising. The growing number of adult and childhood diet-induced obesity necessitates the development of novel strategies to prevent and treat obesity and associated comorbidities.

While various pharmacological treatments are available to help reduce obesity, models for testing new methodologies often lack specific desirable characteristics. There are no approaches available for knocking in TRPV1 at specific tissues/organs in TRPV1 knockout mice.

Therefore, there is a need for a model available for knocking in TRPV1 at specific tissues/organs in TRPV1 knockout mice and methods for producing such models.

### SUMMARY

The disclosure provides, inter alia, TRPV1 knock-in mouse and mouse lines useful for the study of TRPV1-mediated physiology and for the development of pharmacotherapies targeting, for example, obesity and metabolic disease.

In some embodiments, a transgenic mouse is provided. The transgenic mouse's genetic material includes a Cre mouse TRPV1 protein knockout and a rat TRPV1 cyanreporter protein expressed in a tissue.

In other embodiments, a transgenic mouse is provided. The transgenic mouse's genetic material includes a Cre mouse TRPV1 protein knockout and a rat TRPV1 cyanreporter protein expressed in adipose tissue.

In other embodiments, a method for producing a conditional TRPV1 knock-in is provided. The method includes mating a Cre mouse with a mouse expressing a rat TRPV1 and cyanreporter protein resulting in a first progeny comprising mice heterozygous for a Cre allele and heterozygous for a rat TRPV1 and cyanreporter allele; mating the first heterozygous mouse with a mouse TRPV1 knockout resulting in a second progeny; mating the second progeny with a mouse TRPV1 knockout; and obtaining a third progeny comprising mice heterozygous for the TRPV1 knockout allele, the Cre allele, and the rat TRPV1 and cyanreporter allele.

Other and further embodiments are described below.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, for the disclosure may admit to other equally effective embodiments.

FIG. 1 shows a breeding strategy for adipocyte knock-in of TRPV1 according to some embodiments.

To facilitate understanding, identical reference numerals have been used, where possible, to designate identical elements that are common to the FIGURES. It is contemplated that elements and features of one embodiment may be beneficially incorporated in other embodiments without further recitation.

### DETAILED DESCRIPTION

The present disclosure relates to genetically modified (transgenic) non-human eukaryotic animals (e.g., mice) whose germ cells and somatic cells contain a genetic sequence that expresses a TRPV1 protein in specific tissues (e.g., adipose, brain, skeletal muscle, and liver). The genetic sequence is introduced into the animal or into an ancestor of the animal. Advantageously, by using the mouse strain provided herein, interference from TRPV1 proteins being expressed globally in various tissues is prevented. As such, the specific effects of TRPV1 in a specific tissue (e.g., adipose) can be studied and targeted therapies can be found by using the transgenic eukaryotic animal model disclosed herein.

The animal models of the present disclosure can be used to test and develop drug therapies that can counteract, for example, obesity and metabolic diseases, which play causal roles in type 2 diabetes, dyslipidemia, hypertension, and cardiovascular disease. Moreover, the animal models can be used to characterize and determine the role of capsaicin (CAP) in countering high fat diet (HFD)-induced obesity, to elucidate mechanisms of TRPV1 protein expression, and for breeding.

Effective strategies for healthy weight loss (and fat loss) include caloric restriction and/or increasing energy expenditure. In the body, specific tissues are implicated in storing fat and burning fat. White adipose tissue (WAT) stores excess energy as fat, and brown adipose tissue (BAT) burns fat into energy.

Transient receptor potential vanilloid subfamily 1 (TRPV1) protein is a protein implicated in a HFD-induced obesity. TRPV1 activation in cultured adipocytic cell lines is known to induce a browning phenotype in white adipocytes. Also, TRPV1 activation is known to reduce (and/or prevent) obesity by activating central and peripheral mechanisms that regulate metabolism and thermogenesis. Thus, generating mice that express TRPV1 specifically in adipose tissues will aid in understanding the role of TRPV1 in obesity and to develop pharmacotherapies.

While the use of isolated cell lines (i.e., an in vitro system) is helpful in understanding the physiological role of various genes and the proteins they give rise to, more complete information can be obtained by studying the role of these proteins directly in a mammal (i.e., an in vivo system). To this end, various classes of mammals are discussed herein that have altered levels of expression of certain genes. One class of these mammals is so-called transgenic mammals. These mammals have a novel gene or genes, originating from a different species, introduced into their intact genome (genetic material). Another class of mammals is the “knock-in” mammals. Knock-in mammals have one of their own genes deleted and replaced by a variant of that same gene. A combination of the first two techniques can be used to create a transgenic-knock-in mammal that expresses a foreign gene in the locus of the endogenous host gene; such as a rat gene in the mouse locus of the equivalent gene. Another class of mammals is a global null mutant, or so-called “knockout” mammals, wherein expression of an endogenous gene has been suppressed through genetic manipulation, whether by using recombinant or classical genetic techniques. Thus, in knock-out mice, a gene is inactivated so that it is no longer functional, whereas in knock-in mice, exogenous DNA is added to the genome (genetic material) to modify genetic expression.

The present disclosure is exemplified with mouse models. Primate models may be more relevant to human diseases but are more expensive. However, gene editing technology CRISPR-CAS9 has been successfully carried out in a Rhesus monkey and that could be applied to the TRPV1 gene locus in a primate model. As such, other animal models are encompassed within the present disclosure.

The transgenic mice are produced by a site-specific recombination using Cre-Lox recombination technology that involves the targeting and splicing out of a specific gene with the help of a recombinase. Cre is expressed in a specific cell type, creating a cell-type specific deletion of the targeted gene. This method includes mating Cre mice and floxed (sandwich the targeted gene with loxP sequences, e.g., a floxed TRPV1 gene) mice to produce knock-in mice with the targeted gene replaced in certain cell types.

Various Cre mouse models can be used for breeding. The Cre mouse is an example of a mouse system that consists of a single enzyme, Cre recombinase that recombines that sequence without having to insert any extra supporting sequences. Another system that is useful for such creations is the FLP-FRT recombination system. Those of ordinary skill in the art are well aware of other such systems.

It is an object of the present disclosure to provide an animal model having additions of the gene that expresses a rat TRPV1 protein. It is a further object of the present disclosure to provide an animal model comprising a floxed gene that expresses TRPV1. The mouse is one of the animals useful as the animal model of the present disclosure. Another object of the present disclosure is to provide transgenic mice by crossing TRPV1-floxed mice with other mouse models, such as Cre gene related mice.

### Preparation of Knock-in/Transgenic Mammals

The present disclosure relates to transgenic mammals (e.g., mice) and methods of preparing transgenic mammals (e.g., mice). In some embodiments, the transgenic mammal's genetic material comprises a Cre mouse TRPV1 protein knockout; and a rat TRPV1 cyanreporter protein expressed in a specific tissue. The transgenic mammals (e.g., mice) may be prepared as described below.

Mammals containing the knock-in construct and/or transgene can be prepared in any of several ways. The manner of preparation is to generate a series of mammals, e.g., a mouse, each containing one of the desired knock-in constructs or transgenes, as described herein. Such mammals are bred together through a series of crosses, backcrosses and selections, to ultimately generate a single mammal containing all desired knock-in constructs and/or transgenes, where the mammal is otherwise congenic (genetically identical) to the wild type except for the presence of the knock-in(s) constructs and/or transgene(s).

Typically, crossing and backcrossing is accomplished by mating siblings or a parental strain with an offspring, depending on the goal of each particular step in the breeding process. In certain cases, it may be necessary to generate a large number of offspring in order to generate a single offspring that contains each of the knock-in constructs and/or transgenes in the proper chromosomal location. In addition, it may be necessary to cross or backcross over several generations to ultimately obtain the desired genotype.

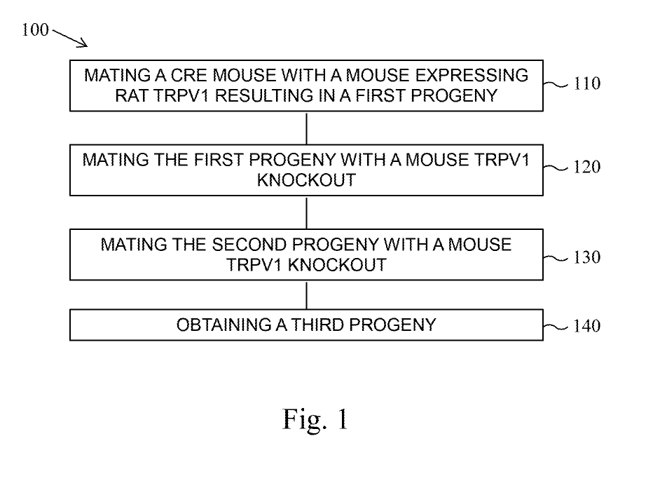

FIG. 1 shows a method 100 of knocking in TRPV1 in the white adipose tissue (WAT) and brown adipose tissue (BAT) of a TRPV1^{-/-} mouse, according to some embodiments. It is contemplated that this method may be performed for other specific tissues (including skeletal muscle, liver, and brain tissues). The method produces a mouse strain that will have a rat TRPV1 cyanreporter protein expressed in specific tissues in a mouse that lacks TRPV1 throughout the body.

The method 100 includes crossing (i.e., mating) a Cre mouse with a mouse having a rat TRPV1 cyanreporter protein at operation 110. The Cre mouse has a Fabp4-Cre transgene which has a mouse Fabp4 promoter directing expression of Cre recombinase. These Fabp4-Cre transgenic mice are a Cre-lox tool useful for deletion of floxed sequences in brown and white adipose tissue and are commercially obtained (Jackson Laboratories). The cross produces a first progeny of four distinct genotypes, including mice heterozygous for the rat TRPV1 and cyanreporter allele and the Cre allele (i.e., Het (rat^{TRPV1} cyanreporter/Cre)), which 25% of the progeny exhibit. Expression of the TRPV1 protein in adipose tissue can be detected by the expression of the cyanfluorescence (cyanreporter) protein.

Method 100 further includes mating the progeny having the Het (rat^{TRPV1} cyanreporter/Cre) with a mouse TRPV1 knock out line (TRPV1^{-/-}) at operation 120. The cross produces a second progeny of four distinct genotypes, including a genotype having Het (rat^{TRPV1} cyanreporter/Cre) and mouse TRPV1 (i.e., Het (rat^{TRPV1} cyanreporter/Cre/mouse^{TRPV1})), which 25% of the progeny exhibit. Method 100 further includes mating the progeny having the Het (rat^{TRPV1} cyanreporter/Cre/mouse^{TRPV1}) with the mouse TRPV1 knock out line (TRPV1^{-/-}) at operation 130. The cross produces a third progeny of eight distinct genotypes (operation 140). One of the genotypes, which 12.5% of the progeny will exhibit, has mice heterozygous for the TRPV1 knock-out, the Cre allele, and the rat TRPV1 allele, i.e., Mouse (rat^{TRPV1} cyanreporter/Cre/mouse^{TRPV1}). Thus, the method generates mice that express rat TRPV1 protein only in the adipose tissue. It is contemplated that the method can be used to express rat TRPV1 protein in other tissue types. In some embodiments, the third progeny is mated with a partner (e.g., a mouse).

In some embodiments, the Cre gene is from a mouse strain. The mouse strain includes B6.129-Trpv1^{tm1(cre)Bbm}/J; B6.129X1-Trpv1^{tm1Jul}/J; B6.129-Trpv1^{tm2Bbm}/J; B6.129P2-Gt(ROSA)26Sor^{tm1(Trpv1,ECFP)Mde}/J; and C57BL/6N-Tg(Trpv1)5917Jsmn/J. Moreover, any tissue-specific Cre to express rat TRPV1 in that specific tissue can be used. For example, breeding with FABP Cre expresses rat TRPV1 in the adipose tissues of TRPV1 knockout mice.

The Cre mouse has a tissue specific transgene, for example an adipose tissue specific transgene, a brain tissue specific transgene, a skeletal muscle tissue specific transgene, a liver tissue specific transgene, a brown adipose tissue specific transgene, and a white adipose tissue specific transgene.

The mice exhibiting the conditional knock-in of rat TRPV1 are valuable in analyzing tissue-specific effects of TRPV1 since these mice will not express TRPV1 in neurons or other tissues. The expression of TRPV1 protein in adipose tissue can also easily be recognized by the expression of the cyan fluorescence protein.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue (about 1 cm is removed from the tip of the tail) and analyzed by Southern analysis or polymerase chain reaction (PCR) for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include suitable biochemical assays such as enzyme and/or immunological assays, histological stains for particular markers or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic mammals may be obtained by mating the transgenic mammal with a suitable partner, or by in vitro fertilization of eggs and/or sperm obtained from the transgenic mammal. Where mating with a partner is to be performed, the partner may or may not be transgenic and/or a knockout; where the partner is transgenic, the partner may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

### Uses of Knock-in Mammals

In general, knock-in mammals have a variety of uses depending on the gene or genes that have been replaced. For example, where the gene or genes replaced encode proteins believed to be involved in obesity, the mammal may be used to screen for drugs useful for obesity modulation, i.e., drugs that either enhance or inhibit these activities. The tissue specific TRPV1 knock-in mice of the present disclosure can be used to screen potential drugs for the treatments of obesity and metabolic disorders. Screening for useful drugs would involve administering the candidate drug over a range of doses to the mouse, and assaying at various time points for the effect(s) of the drug on the disorder being evaluated.

In addition, mammals of the present disclosure can be useful for studying the effects of particular TRPV1 gene mutations. Embodiments of the TRPV1 knock-in mice and the progeny of this disclosure will also have a variety of uses depending on the additional transgenes that can be expressed and/or the knock-in constructs they may contain. Screening for a useful drug involves first inducing the disease, or inducing a model of the disease, in the mammal and then administering the candidate drug over a range of doses to the mammal, and assaying at various time points for the effect(s) of the drug on the disease or disorder being evaluated. Alternatively, or additionally, the drug is administered prior to or simultaneously with exposure to induction of the disease or disease model. In other embodiments, the TRPV1 knock-in mice are further useful for drug research and development, for example, in in vivo protocols to distinguish on-target/off-target effects.

In addition to screening a drug for use in treating a disease or condition, the mammal of the present disclosure is useful in designing a therapeutic regimen aimed at preventing or curing the disease or condition. For example, the mammal might be treated with a combination of a particular diet, exercise routine, radiation treatment, and/or one or more compounds or substances either prior to, or simultaneously, or after, the onset of the disease or condition. Such an overall therapy or regimen might be more effective at combating the disease or condition than treatment with a compound alone. In addition, such criteria as blood pressure, body temperature, body weight, pulse, behavior, appearance of coat (ruffled fur) and the like could be evaluated.

The TRPV1 knock-in mice of this disclosure may also be used to generate one or more cell lines. Such cell lines have many uses, as for example, to evaluate the effect(s) of the knock-in on a particular tissue or organ, and to screen compounds that may affect the level of activity of the TRPV1 in the tissue. Such compounds may be useful as therapeutics.

Production of such cell lines may be accomplished using a variety of methods, known to the skilled artisan. The actual culturing conditions will depend on the tissue and type of cells to be cultured. Various media containing different concentrations of macro and micro nutrients, growth factors, serum, and the like, can be tested on the cells without undue experimentation to determine the conditions for growth and proliferation of the cells. Similarly, other culturing conditions such as cell density, media temperature, and carbon dioxide concentrations in the incubator can also readily be evaluated and determined, and identifying compounds that affect this process.

The effect of dietary capsaicin (CAP) can also be studied using the mouse strains disclosed herein. It is believed that knocking in TRPV1 specifically in the adipose tissues of TRPV1^{-/-} mice will confer CAP sensitivity to these mice and CAP will counter HFD-induced obesity in these mice.

The present disclosure provides TRPV1 knock-in mouse and mouse lines useful for the study of TRPV1-mediated physiology and for the development of pharmacotherapies targeting, for example, obesity and metabolic disease.

The descriptions of the various embodiments of the present disclosure have been presented for purposes of illustration, but are not intended to be exhaustive or limited to the embodiments disclosed. While the foregoing is directed to embodiments of the present disclosure, other and further embodiments of the disclosure may be devised without departing from the basic scope thereof, and the scope thereof is determined by the claims that follow. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A transgenic mouse whose genetic material comprises:
a Cre mouse TRPV1 protein knockout; and
a rat TRPV1 cyanreporter protein expressed in a tissue.

2. The transgenic mouse of claim 1, further comprising:
a floxed TRPV1 gene.

3. The transgenic mouse of claim 1, wherein the Cre mouse TRPV1 has a tissue specific transgene, wherein the tissue specific transgene is selected from the group consisting of adipose tissue specific transgene, brain tissue specific transgene, skeletal muscle tissue specific transgene, and liver tissue specific transgene.

4. The transgenic mouse of claim 1, wherein the tissue is brown adipose tissue.

5. The transgenic mouse of claim 1, wherein the Cre mouse TRPV1 has an adipose tissue specific transgene.

6. The transgenic mouse of claim 1, wherein the Cre mouse TRPV1 has a Cre gene selected from mouse strain
B6.129-Trpv1^{tm1(cre)Bbm}/J;
B6.129X1-Trpv1^{tm1Jul}/J;
B6.129-Trpv1^{tm2Bbm}/J;
B6;129P2-Gt(ROSA)26Sor^{tm1(Trpv1,ECFP)Mde}/J; and
C57BL/6N-Tg(Trpv1)5917Jsmn/J.

7. The transgenic mouse of claim 1, wherein the Cre mouse TRPV1 has a Cre gene selected from the mouse strain B6.129-Trpv1^{tm1(cre)Bbm}/J.

8. A transgenic mouse whose genetic material comprises:
a Cre mouse TRPV1 protein knockout; and
a rat TRPV1 cyanreporter protein expressed in adipose tissue.

9. The transgenic mouse of claim 8, further comprising:
a floxed TRPV1 gene.

10. The transgenic mouse of claim 8, wherein the adipose tissue is white adipose tissue.

11. The transgenic mouse of claim 8, wherein the adipose tissue is brown adipose tissue.

12. The transgenic mouse of claim 8, wherein the Cre mouse TRPV1 has a Cre gene selected from the mouse strain
B6.129-Trpv1^{tm1(cre)Bbm}/J;
B6.129X1-Trpv1^{tm1Jul}/J;
B6.129-Trpv1^{tm2Bbm}/J;
B6;129P2-Gt(ROSA)26Sor^{tm1(Trpv1,ECFP)Mde}/J; and
C57BL/6N-Tg(Trpv1)5917Jsmn/J.

13. The transgenic mouse of claim 8, wherein the Cre mouse TRPV1 has a Cre gene selected from the mouse strain B6.129-Trpv1^{tm1(cre)Bbm}/J.

14. A method for producing a conditional TRPV1 knock-in comprising:
mating a Cre mouse with a mouse expressing a rat TRPV1 and cyanreporter protein resulting in a first progeny comprising mice heterozygous for a Cre allele and heterozygous for a rat TRPV1 and cyanreporter allele;
mating the first heterozygous mouse with a mouse TRPV1 knockout resulting in a second progeny;
mating the second progeny with a mouse TRPV1 knockout; and
obtaining a third progeny comprising mice heterozygous for a TRPV1 knockout allele, the Cre allele, and the rat TRPV1 and cyanreporter allele.

15. The method of claim 14, wherein a Cre gene of the Cre mouse is selected from the mouse strain
B6.129-Trpv1^{tm1(cre)Bbm}/J;
B6.129X1-Trpv1^{tm1Jul}/J;
B6.129-Trpv1^{tm2Bbm}/J;
B6;129P2-Gt(ROSA)26Sor^{tm1(Trpv1,ECFP)Mde}/J; and
C57BL/6N-Tg(Trpv1)5917Jsmn/J.

16. The method of claim 14, wherein the Cre mouse TRPV1 has a Cre gene selected from the mouse strain B6.129-Trpv1^{tm1(cre)Bbm}/J.

17. The method of claim 14, wherein the Cre mouse has a Fabp4 promoter directing expression of Cre recombinase.

18. The method of claim 14, wherein the Cre mouse has a tissue specific transgene, wherein the tissue specific transgene is selected from the group consisting of adipose tissue specific transgene, brain tissue specific transgene, skeletal muscle tissue specific transgene, and liver tissue specific transgene.

19. The method of claim 14, wherein the Cre mouse has an adipose tissue specific transgene.

20. The method of claim 14, further comprising mating the third progeny with a partner.
